(19) United States
(12) Patent Application Publication
(10) Pub. No.: US 2008/0233075 A1
(43) Pub. Date: Sep. 25, 2008

(54) COSMETIC COMPOSITION FOR SKIN TIGHTENING

(76) Inventors: Marina Sokolinsky, Smithtown, NY (US); Krisztina Toth, Floral Park, NY (US); Peter Lentini, West Babylon, NY (US); Geoffrey Hawkins, Yardley, PA (US); Ken Marenus, Dix Hills, NY (US)

Correspondence Address: Mimi Yang, Suite 345 South, 155 Pinelawn Road, Melville, NY, 11747, US

(21) Appl. No.: 11/689,571

(22) Filed: Mar. 22, 2007

(51) Int. Cl. A61K 8/64; A61K 8/65

(52) U.S. Cl. 424/78.03

(57) ABSTRACT

The present invention provides a topical composition that contains a water-soluble film-forming polymer, a bimodal copolymer comprising a first polymeric component with anionic functional groups and a second polymeric component with cationic functional groups, and one or more biological polymers that are derived from a source selected from the group consisting of animals, plants, algae, fungi, and bacteria or are biotechnologically synthesized. The first and second polymeric components of the bimodal copolymer form an interpenetrating polymeric network that interacts with the water-soluble film-forming polymer and the biological polymers to form a polymeric film with superior skin-firming and skin-toning effects. Such a topical composition can be applied to saggy or wrinkled skin for enhancing the appearance of the skin.

### FIELD OF THE INVENTION

The present invention relates to cosmetic compositions for application to the skin. More specifically, the present invention relates to cosmetic compositions that enhance the appearance of skin by providing both short-term skin tightening effects and long-term wrinkle reduction effects, as well as other skin benefits.

### BACKGROUND OF THE INVENTION

Skin is subject to damages by many extrinsic and intrinsic factors. Extrinsic factors include ultraviolet radiation (e.g., from sun exposure), environmental pollution, wind, heat, low humidity, harsh chemicals, abrasives, and the like. Intrinsic factors include chronological aging and other biochemical changes occurred within the skin. These extrinsic and intrinsic factors can result in visible signs of skin aging, such as the appearance of deep wrinkles and fine lines on the skin. For many people, wrinkles and fine lines are a reminder of the disappearance of their youth. Therefore, there is an enormous demand for cosmetic formulations that can effectively tighten the skin and reduce the appearance of wrinkles and/or fine lines.

Some cosmetic compositions comprise substances of natural origin, such as plant, egg, milk, or animal derivatives, as skin-tightening agents. For example, serum albumin exhibits a significant skin-tightening effect and has been used to reduce the appearance of wrinkles. However, serum albumin is uncomfortable to use, and it also leaves an unsightly while film on the skin.

Other cosmetic compositions have employed synthetic polymers as the skin-tightening agents. For example, U.S. Pat. No. 4,777,041 describes a wrinkle treatment formulation containing a gelable hydrophilic polyurethane and a precipitated silica thickener gelling agent that fills wrinkles when dried. However, the polyurethane and silica components of this formulation have various undesirable properties that make it unsuitable for widespread use.

Consequently, there is still a continuing need for improved cosmetic compositions that provide a suitable skin-tightening effect and can be used to improve the appearance of the skin, without the drawbacks of conventional skin-tightening compositions or formulations.

### SUMMARY OF THE INVENTION

The composition of the present invention contains at least: (1) a water-soluble film-forming polymer, (2) a bimodal copolymer having at least a first polymeric component comprising anionic functional groups and a second polymeric component comprising cationic functional groups, and (3) one or more biological polymers that are derived from a source selected from the group consisting of animals, plants, algae, fungi, and bacteria or are biotechnologically synthesized.

Preferably, the polymeric film formed by the topical composition of the present invention is characterized by a skin-firming effect of no less than about 30%, more preferably no less than about 50%, and most preferably no less than about 60%. Alternatively or additionally, the so formed polymeric film is characterized by a skin-toning effect of no less than about 50%, more preferably no less than about 100%, and most preferably no less than about 180%. Further, the polymeric film is preferably, but not necessarily, characterized by an adhesion of no less than about 1500 grams, more preferably no less than about 1600 grams, and most preferably no less than 1700 grams.

In another aspect, the present invention relates to a method of enhancing the appearance of skin, comprising applying to the skin a topical composition as described hereinabove. Preferably, but not necessarily, the topical composition is applied to saggy or wrinkled skin. More preferably, the topical composition is chronically applied to the saggy or wrinkled skin for a period of at least one month and at a frequency ranging from about once per week to about five times per day.

Other aspects and objectives of the present invention will become more apparent from the ensuring description, examples, and claims.

### DEFINITION

The term “skin-firming effect” as used herein refers to the percentage increase in skin firmness before and after application of a sample topical composition. The skin firmness indicates the resistance exhibited by the skin against a suction power generated by a negative pressure applied onto the skin through an aperture in a probe of the Cutometer® MPA 580 (manufactured by Courage+Khazaka Electronic GmbH at Germany). Specifically, the skin firmness is calculated as 1/Uf, i.e., inverse to the degree of skin distension (Uf) induced by application of the negative pressure. The skin firmness before application of the sample topical composition is herein designated as “Fb,” and the skin firmness after application of the sample topical composition is herein designated as “Fa.” The skin-firming effect (F) is then calculated as (Fa−Fb)/Fb.

The term “skin-toning effect” as used herein refers to the percentage increase in skin tonicity before and after application of the sample topical composition. The skin tonicity indicates the ability of the skin to return to its original state after the negative pressure is removed, which can also be measured by the Cutometer® MPA 580. Specifically, the skin tonicity is calculated as 1/Ua, i.e., inverse the degree of skin deformation (Ua) remained after the negative pressure is removed. The skin tonicity before application of the sample topical composition is herein designated as “Tb,” and the skin tonicity after application of the sample topical composition is herein designated as “Ta.” The skin-toning effect (T) is then calculated as (Ta−Tb)/Tb.

The term “adhesion” as used herein refers to the adhesion (in terms of grams) exhibited by a film applied onto a glass surface, as measured by a Texture Analyzer (model # TA XT PLUS) manufactured by Texture Technology Corporation at Scardale, N.Y.

The term “water-soluble” as used herein refers to the solubility of a material of not less than 270 g/l in water at a temperature of about 25° C.

### BRIEF DESCRIPTION OF THE DRAWINGS

FIG. 1 is a bar chart illustrating the skin firmness (1/Uf) observed after application of a topical composition formulated according to one embodiment of the present invention, in comparison with those observed after application of various individual ingredients contained therein.

FIG. 2 is a bar chart illustrating the skin tonicity (1/Ua) observed after application of the same topical composition as in FIG. 1, in comparison with those observed after application of various individual ingredients contained therein.

FIG. 3 is a bar chart illustrating the skin firmness (1/Uf) observed after application of a topical composition formulated according to another embodiment of the present invention, in comparison with those observed after application of two skin-tightening products currently available on the market (i.e., conventional skin-tightening products).

FIG. 4 is a bar chart illustrating the skin tonicity (1/Ua) observed after application of the same topical composition as in FIG. 3, in comparison with those observed after application of the conventional skin-tightening products.

### DETAILED DESCRIPTION OF THE INVENTION AND PREFERRED EMBODIMENTS THEREOF

Inventors of the present invention have discovered, surprisingly and unexpectedly, that by combining a bimodal copolymer (which will be described in greater detail hereinafter) with a water-soluble film-forming polymer and one or more biological polymers, a polymeric film of remarkably improved physical properties can be formed on the skin. While not wanting to be bound by any theories, it is believed that the bimodal copolymer forms an interpenetrating polymeric network that synergistically interacts with the water-soluble film-forming polymer and the biological polymers to achieve superior skin-firming and skin-toning effects, enhanced flexibility and good skin-adhesion, which have not been observed in any other skin-tightening product that is currently available on the market. It is believed that they can only be achieved by the simultaneous presence of and the synergic interactions between the water-soluble film-forming polymer, the bimodal polymer, and the biological polymers.

The water-soluble film-forming polymer functions as the main skin-firming agent in the compositions of the present invention. Suitable water-soluble synthetic film-forming polymers that can be used in the present invention include, but are not limited to: polyvinylpyrrolidone (PVP), polyvinylacetate (PVA), PVP/PVA copolymers, carboxymethylcellulose (CMC), cellulosic ethers, polyamides, acrylates polymers and copolymers, PVP/dimethylaminopropylamine (DMAPA) acrylates copolymers, vinylpyrrolidone (VP)/lauryl methacrylate (LMA) copolymers, ammonium acrylodimethyltaurate/VP copolymers, isobutylene (IB)/maleic anhydride (MA) copolymers (such as imidized IB/MA copolymers), acrylates/acrylonitrogen copolymers, polysiloxanes and other silicon-containing polymers. Note that more than one water-soluble synthetic film-forming polymer can be used conjunctively in the topical composition of the present invention.

In a particular preferred embodiment of the present invention, the water-soluble film-forming polymer comprises PVP with an average molecular weight ranging from about 25,000 to about 5,000,000.

The water-soluble film-forming polymer may be provided in the topical composition of the present application in an amount ranging from about 0.1% to about 20%, preferably from about 0.5% to about 15%, more preferably from about 1% to about 10%, and most preferably from about 3% to about 7%, by total weight of the composition.

The bimodal copolymer in the topical form functions as a polymeric matrix for supporting and integrating the water-soluble film-forming polymer with the biological polymers. The term “bimodal” as used herein refers to polymers or copolymers having at least two polymeric components, the first of which contains anionic functional groups and the second of which contains cationic functional groups.

The bimodal copolymer used in the topical composition of the present invention is preferably a copolymer that comprises a first polymeric component having one or more monomeric units with the following formula:

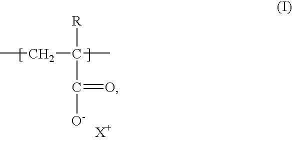

wherein R is hydrogen or an alkyl group and X⁺ is a salt-forming cation, and a second polymeric component having one or more monomeric units with the following formula:

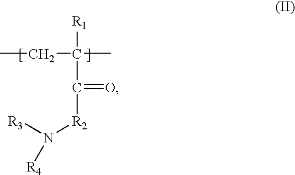

or a quatemized adduct thereof, wherein R₁, R₃ and R₄ are, independently, hydrogen or an alkyl group and R₂ is an alkyl group. More preferably, the first polymeric component of the bimodal copolymer comprises one or more monomeric acrylate units, and the second polymeric component comprises one or more monomeric dialkylaminoalkylmethacrylate units or quaternized adducts thereof. More preferably, the bimodal copolymer comprises Syntran® PC5100 available from Interpolymer Corporation at Canton, Mass. For more detailed description of the bimodal copolymer, see WO 2005/087191 A1, the content of which is incorporated herein by reference in its entirety for all purposes.

The bimodal copolymer as described hereinabove may present in the topical composition of the present invention at an amount ranging from about 0.1% to about 20%, preferably from about 0.5% to about 15%, more preferably from about 1% to about 10%, and most preferably from about 2% to about 8% by total weight of the composition.

The biological polymers as used in the present invention function as plasticizers for the water-soluble film-forming polymer and the bimodal polymer. Unlike synthetic plasticizers (such as glycol, ethylene glycol, propylene glycol, and the like) that typically form a tacky film that dries very slowly, the biological polymers of the present invention help to achieve a sufficiently flexible, but significantly less tacky polymeric film that dries within a shortened period of time. In addition, the biological polymers may provide long term benefits to the skin. Such biological polymers can be any type of film-forming proteins, polysaccharides, or other naturally occurring polymers that exhibit long-term skin benefits. For example, the biological polymers of the present invention can be derived from animals, plants, fungi, algae or bacteria or can be biotechnologically synthesized. Suitable biological polymers that can be used for practicing the present invention include, but are not limited to: keratin, cellulose gum, alginates (such as sodium alginate), Chondrus crispus extract (carrageenans), sweet almond proteins (e.g., Polylift® from Silab), Argania spinosa (i.e., argan) proteins (e.g., Argatensyl™ from Laboratoires Sérobiologiques), corn proteins, wheat proteins (e.g., Tritisol™ from Croda, Inc.), silk proteins (e.g., Silk Crystal™ gel from Charles B. Chrystal Co. Inc.), milk proteins (e.g., Lactofirm™ from Barnet Products), soy proteins, tobacco leaf proteins, bacterial proteins, fibrous proteins derived from beans, egg white proteins (e.g., albumins), and baterial polysaccharides (such as Fucogel or water/biosaccharide gum-1 from Solabia). Particularly preferred biological polymer is keratin, such as hydrolyzed keratin.

Preferably, the biological polymers are selected from the group consisting of keratin, cellulose gum, sodium alginate, and carrageenans, which exhibit long-term skin-firming and skin-toning effects. More preferably, the compositions of the present invention comprise two or more of such biological polymers, and most preferably, such compositions are essentially free of synthetic plasticizers (i.e., less than 1% by weight of the total composition).

The biological polymers as described hereinabove may present in the topical composition of the present invention at an amount ranging from about 0.05% to about 60%, preferably from about 0.1% to about 30%, more preferably from about 0.5% to about 10% by total weight of the composition.

Although not wishing to be bound by any specific theory, it is believed that the above-described water-soluble film-forming polymer, bimodal polymer, and biological polymer interact with one another in a synergistic manner to form a polymeric film with significantly improved physical properties. Specifically, it is believed that the first and the second polymeric components of the bimodal copolymer form an interpenetrating polymer network that binds to and interacts with both the water-soluble film-forming polymer and the biological polymers, which consequently modifies/optimizes the performance of such polymers and leads to synergistic changes in the properties of the resulting polymeric film. For example, addition of the bimodal copolymer into the water-soluble film-forming polymer and the biological polymers results in a polymeric film that has a skin-firming effect (F) and a skin-toning effect (T) that are significantly higher than those exhibited by polymeric films formed of the individual components, i.e., the bimodal copolymer itself, the water-soluble film-forming polymer, and the biological polymers, as shown in Example 2 hereinafter. Further, the skin-firming effect (F) and the skin-toning effect (T) exhibited by the polymeric film formed by the topical composition of the present invention are significantly higher than that exhibited by other skin-tightening cosmetic products that are currently available on the market, as shown in Example 2. Such a polymeric film also exhibits improved good adhesion properties sufficient for topical applications.

The topical composition of the present invention may further contain one or more skin care active ingredients or skin care actives. The term “skin care active ingredients” or “skin care actives” as used herein refers to agents that provide benefits to the skin rather than merely improving the physical characteristics of the topical composition. For example, the topical composition may comprise anti-aging agents that are capable of protecting the skin against photo- or chrono-aging by scavenging free radicals, preventing lipid peroxidation, inactivating lipogenase, inhibiting undesired enzymatic activities, and stimulating collagen synthesis. The topical composition may also include anti-acne agents, enzyme-inhibiting agents, collagen-stimulating agents, sunscreen agents, antioxidant, exfoliants, agents for the eradication of age spots, keratoses and wrinkles, analgesics, anesthetics, antibacterials, antiyeast agents, antifungal agents, antiviral agents, antidandruff agents, antidermatitis agents, antipruritic agents, antiemetics, anti-inflammatory agents, antihyperkeratolytic agents, antiperspirants, antipsoriatic agents, antiseborrheic agents, antiwrinkle agents, antihistamine agents, skin lightening agents, depigmenting agents, vitamins, corticosteroids, self-tanning agents, hormones, retinoids such as retinoic acid and retinol, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, topical steroids such as hydrocortisone, hydrocortisone 21-acetate, hydrocortisone 17-valerate, and hydrocortisone 17-butyrate, betamethasone valerate, betamethasone diproprionate, benzoyl peroxide, crotamiton, propranolol, promethazine, vitamin A palmitate, vitamin E acetate and mixtures thereof.

The above-described skin care active ingredients are only optional components of the topical composition of the present invention and may be omitted from such composition without materially affecting the skin tightening function of the topical composition.

The topical composition of the present application further comprises a cosmetically acceptable vehicle. For purpose of the present invention, cosmetically acceptable vehicles are substances that can be used to formulate the above-described active ingredients into a cream, gel, emulsion, liquid, suspension, nail coating, skin oil, or lotion that can be topically applied. Substances which may be formulated into the topical composition of the present application include, but are not limited to: moisturizing agents, astringent agents, chelating agents, surfactants, emollients, preservatives, stabilizers, thickeners, humectants, pigments, etc. Preferably, but not necessarily, such vehicles aid the formation of a protective layer on the skin. The vehicle or vehicles can present in the topical composition of the present invention at an amount ranging from about 1% to about 99.9%, preferably from about 50% to about 99.5%, more preferably from about 70% to about 99%, and most preferably from about 80% to 90% by total weight of the topical composition.

For example, emollients which may be used in the topical composition of the present invention include, but are not limited to: stearyl alcohol, cetyl alcohol, oleyl alcohol, isocetyl alcohol, fatty alcohols, propane-1,2-diol, butane-1,3-diol, octadecan-2-ol, glyceryl monostearate, isopropyl isostearate, stearic acid, isostearic acid, isocetyl stearate, isopropyl stearate, butyl stearate, isopropyl laurate, hexyl laurate, decyl oleate, isobutyl palmitate, cetyl palmitate, isopropyl palmitate, palmitic acid, dimethylpolysiloxane, glyceryl monoricinoleate, di-n-butyl sebacate, isopropyl myristate, butyl myristate, myristyl myristate, isopropyl linoleate, lauryl lactate, myristyl lactate, polyethylene glycol, triethylene glycol, lanoline, acetylated lanolin, sesame oil, coconut oil, arrachis oil, castor oil, mink oil, mineral oil, and petroleum.

A variety of water soluble preservatives can be added to the topical compositions of the present invention to provide a prolonged shelf life. Suitable preservatives include, but are not limited to: potassium sorbate, imidazolidinyl urea, p-hydroxy benzoate, esters of p-hydroxybenzoic acid, CTFA designation parabens, ethylhexylglycerin, caprylyl glycol/phenoxyethanol/hexylene glycol, etc. Other preservatives suitable for use in the topical compositions of the present invention are disclosed in the International Cosmetic Ingredient Dictionary and Handbook, twelfth edition, 2004, the entire disclosure of which is herein incorporated by reference.

Further, any suitable thickening agent commonly used in cosmetic and personal care products can be added to the topical compositions of the present invention to improve the viscosity of the compositions. Preferably, the thickening agent includes, but is not limited to: starch, gum (such as gum arabic), clay (such as kaolin), hydrated aluminum silicate, magnesium aluminum silicate, fumed silica, polyacrylic acid, hydroxyehtylcellulose, carboxyvinyl polymer, sodium carboxymethyl cellulose or other cellulose derivatives, ethylene glycol monostearate and sodium alginates.

Humectants which may be used include, but are not limited to: polyhydric alcohols including glycerol, polyalkylene glycols, and alkylene polyols and mixtures thereof, hyaluronic acid, urea, glycerin, sorbitol, sodium 2-pyrrolidone-5-carboxylate, soluble collagen, dibutylphthalate and gelatin.

The topical composition of the present invention may also contain additional cosmetic ingredients that add to the aesthetics of performance of the present invention. For example, pigments, powders and fragrances may be used to increase the aesthetic appeal of the present invention.

The topical compositions optionally include one or more colorants or pigments selected from cosmetically acceptable inorganic and organic pigments, such as those disclosed in the International Cosmetic Ingredient Dictionary and Handbook, twelfth edition, 2004. The inorganic pigments may include iron oxides, titanium dioxide, zinc oxide, metal silicates (such as micas, talc, kaolin, etc.), bismuth oxychloride and the like. The organic pigments may include barium lake, calcium lake, aluminum lake, zirconium lake, calcium lake, D&C and FD&C colors and lakes thereof. The pigment(s), if present in the compositions of the present invention, is preferably present in a total amount from about 1% to about 20%, and more preferably from about 5% to about 15% by total weight of the composition.

Powders that can be added into the topical composition of the present invention include chalk, talc, fuller's earth, colloidal silicon dioxide, sodium polyacrylate, tetra alkyl and/or trialkyl ammonium smectites, and chemically modified magnesium aluminum silicate.

The topical composition of the present invention may optionally comprise a fragrance in an amount sufficient to make the composition more appealing to the consumer. Preferably, the fragrance is in the amount of from about 0.01% to about 10% by total weight of the composition.

The above-described topical compositions are particularly useful as skin-tightening products for improving the appearance of skin. Such topical compositions can be applied to eyes, chin, neck and other facial areas to reduce sagginess of the skin and appearance of any wrinkle or fine line therearound, and it can also be applied to other bodily areas containing saggy or wrinkled skin. The resulting polymeric film provides immediate, visible skin-tightening and wrinkle-reduction effects. It is effective in reducing both fine lines and deeper, greatly visible wrinkles. Further, the polymer film is bio-compatible and comfortable to wear, and can therefore be left on the skin for a relatively long period of time to provide long-term skin benefits.

Further, the topical compositions of the present invention can be used for regulating other skin disorders with the addition of one or more actives effective for preventing, retarding, arresting, or treating such other skin disorders. For example, the topical compositions of the present invention may be used for regulating dry skin, xerosis, dandruff, keratoses, psoriasis, eczema, age spots, lentigines, melasmas, blemished skin, hyperpigmentation, hyperkeratoses, inflammatory dermatoses, and age-related skin changes.

The methods of application in the present invention will depend on the ultimate intended use of composition. The topical composition can be applied locally to the saggy or wrinkled skin, or it can be applied to the entire body of the user. The topical composition of the present invention may be applied to the skin on an as-needed basis, to achieve irunediate wrinkle reduction results (typically observable within five or ten minutes). Alternatively, the topical composition can be applied to the skin repeatedly according to a pre-set schedule. The topical composition of the present invention may be applied directly to clean skin, before application of any moisturizer, foundation, make-up, etc. Alternatively, the topical composition of the present invention can be applied over moisturizer, and optionally over foundation and/or make-up. The amount of the topical composition applied each time, the area of application, the duration of application, and the frequency of application can vary widely, depending on the specific need of the user. For example, the topical composition can be applied for a period of at least one month and at a frequency ranging from about once per week to about five times per day. For another example, the topical composition is applied for a period of about six months and at a frequency ranging from about three times a week to about three times per day, and preferably about once or twice per day. The topical composition may comprise the active components at a total amount ranging from about 0.01% to about 90%, preferably from about 1% to about 20%, and more preferably from about 1% to about 5%. However, it should be noted that it is well within the purview of the skilled artisan, such as a dermatologist or other health care provider, to tailor the dosages of the topical compositions of the present invention according to specific patient needs.

The following examples further illustrate various specific embodiments of the present invention, without limiting the broad scope thereof.

### EXAMPLE 1

### Skin-Tightening Compositions

| | | |
|---|---|---|
| Phases | Components | Wt % |

Formula I

| | | |
|---|---|---|
| Phase A | Purified water | 55.75 |
| | Disodium EDTA | 0.10 |
| | Potassium sorbate | 0.10 |
| | KGel (hydrolyzed keratin/water/phenoxyethanol) | 4.50 |
| | Biopeptide EL (glyceryl polymethacrylate/PEG-8/palmitoyl | 0.50 |
| | oligopeptide) | |
| | Phenoxyethanol | 0.75 |
| Phase B | PVP | 4.00 |
| Phase C | Syntran PC5100 NP (water/ammonium acrylate copolymer/1,3- | 3.00 |
| | butanediol/C₁₁–C₁₅ pareth-7/sodium laureth-12 sulfate/phenoxyethanol) | |
| Phase D | Purified water | 10.00 |
| | Carrageenan | 0.10 |
| Phase E | Purified water | 10.00 |
| | Cellulose gum | 0.50 |
| Phase F | Purified water | 10.00 |
| | Sodium alginate | 0.50 |
| Phase G | Larch tree extract | 0.20 |

Formula II

| | | |
|---|---|---|
| Phase A | Purified water | 43.50 |
| | Disodium EDTA | 0.10 |
| | Potassium sorbate | 0.10 |
| | KGel (hydrolyzed keratin/water/phenoxyethanol) | 4.50 |
| | Glycerin | 0.25 |
| | Biopeptide EL (glyceryl polymethacrylate/PEG-8/palmitoyl | 0.50 |
| | oligopeptide) | |
| | Phenoxyethanol | 0.75 |
| | BC Bioconverted I-white birch (saccharomyces lysate | 2.00 |
| | extract/declustered (−) water/betula alba extract) | |
| Phase B | Denatured alcohol | 7.00 |
| | Boswellia serrata extract | 0.50 |
| | Silybum marianum fruit extract | 0.10 |
| Phase C | PVP | 5.00 |
| Phase D | Syntran PC5100 NP (water/ammonium acrylate copolymer/1,3- | 3.00 |
| | butanediol/C₁₁–C₁₅ pareth-7/sodium laureth-12 sulfate/phenoxyethanol) | |
| Phase E | Purified water | 10.00 |
| | Carrageenan | 0.10 |
| Phase F | Purified water | 10.00 |
| | Avicel PC 611 Stabilizer (microcrystalline cellulose/cellulose gum) | 0.90 |
| Phase G | Purified water | 10.00 |
| | Sodium alginate | 0.25 |
| Phase H | Larch tree extract | 0.20 |
| Phase I | Aristoflex AVC (ammonium acrylodimethyltaurate/VP copolymer) | 1.25 |

Formula III

| | | |
|---|---|---|
| Phase A | Purified water | 85.6 |
| | Disodium EDTA | 0.10 |
| | Potassium sorbate | 0.10 |
| | KGel (hydrolyzed keratin/water/phenoxyethanol) | 3.50 |
| | Biopeptide EL (glyceryl polymethacrylate/PEG-8/palmitoyl | 0.50 |
| | oligopeptide) | |
| Phase B | PVP | 4.00 |
| | Syntran PC5100 NP (water/ammonium acrylate copolymer/1,3- | 3.00 |
| | butanediol/C₁₁–C₁₅ pareth-7/sodium laureth-12 sulfate/phenoxyethanol) | |
| Phase C | Algin | 0.50 |
| Phase D | Sodium hyaluronate | 0.10 |
| | Carrageenan | 0.10 |
| Phase E | Cellulose gum | 0.50 |
| Phase F | Phenoxyethanol | 0.75 |
| Phase G | Glycerin | 0.50 |
| Phase H | Aristoflex AVC (ammonium acrylodimethyltaurate/VP copolymer) | 0.75 |

Formula IV

| | | |
|---|---|---|
| Phase A | Purified water | 83.06 |
| | Disodium EDTA | 0.12 |
| | Citric acid | 0.10 |
| | Potassium sorbate | 0.12 |
| | KGel (hydrolyzed keratin/water/phenoxyethanol) | 4.00 |
| | Biopeptide EL (glyceryl polymethacrylate/PEG-8/palmitoyl | 0.50 |
| | oligopeptide) | |
| Phase B | PVP | 5.00 |
| | Syntran PC5100 NP (water/ammonium acrylate copolymer/1,3- | 4.00 |
| | butanediol/C₁₁–C₁₅ pareth-7/sodium laureth-12 sulfate/phenoxyethanol) | |
| Phase C | Phenoxyethanol | 0.85 |
| Phase D | Algin | 0.50 |
| Phase E | Cellulose gum | 0.50 |
| Phase F | Glycerin | 0.50 |
| Phase G | Aristoflex AVC (ammonium acrylodimethyltaurate/VP copolymer) | 0.75 |

Formula V

| | | |
|---|---|---|
| Phase A | Purified water | 82.58 |
| | Disodium EDTA | 0.12 |
| | KGel (hydrolyzed keratin/water/phenoxyethanol) | 4.00 |
| | Biopeptide EL (glyceryl polymethacrylate/PEG-8/palmitoyl | 0.50 |
| | oligopeptide) | |
| | PVP | 5.00 |
| Phase B | Syntran PC5100 NP (water/ammonium acrylate copolymer/1,3- | 4.00 |
| | butanediol/C₁₁–C₁₅ pareth-7/sodium laureth-12 sulfate/phenoxyethanol) | |
| Phase C | Germazide PSG (phenoxyethanol/chlorphenesin/glycerin/sorbic acid) | 1.50 |
| Phase D | Algin | 0.50 |
| Phase E | Citric acid | 0.05 |
| Phase F | Cellulose gum | 0.50 |
| Phase G | Glycerin | 0.50 |
| Phase H | Aristoflex AVC (ammonium acrylodimethyltaurate/VP copolymer) | 0.75 |

FORMULA VI

| | | |
|---|---|---|
| Phase A | Deionized water | 82.105 |
| | Disodium EDTA | 0.12 |
| | Potassium sorbate | 0.15 |
| | KGel (hydrolyzed keratin/water/phenoxyethanol) | 2.40 |
| | PVP | 4.00 |
| Phase B | Syntran PC5100 NP (water/ammonium acrylate copolymer/1,3- | 4.00 |
| | butanediol/C₁₁–C₁₅ pareth-7/sodium laureth-12 sulfate/phenoxyethanol) | |
| Phase C | Algin | 0.40 |
| Phase D | Glycerin | 0.50 |
| Phase E | Acrylates copolymer | 4.00 |
| Phase F | Phenoxyethanol | 0.90 |
| Phase G | Citric acid | 0.075 |
| Phase H | Methylmethacrylate crosspolymer | 0.50 |
| Phase I | Cosmocil CQ (water/polyaminopropyl biguanide) | 0.10 |
| Phase J | Aristoflex AVC (ammonium acrylodimethyltaurate/VP copolymer) | 0.75 |

Various phases as described hereinabove in Formulas I-VI were mixed together in a stepwise manner by a rotary mixer and/or a propeller mixer at approximately 25° C. to form a homogenous batch.

### EXAMPLE 2

### Skin-Firming and Skin-Toning Effects

Firmness and tonicity of the skin after application of various sample compositions were measured using the Cutometer® MPA 580 (from Courage+Khazaka Electronic GmbH in Germany).

The following is a list of various sample compositions tested in this experiment:
Sample 1: an aqueous composition containing 4 wt % of PVP in de-ionized water;
Sample 2: an aqueous composition containing 0.75 wt % of Aristoflex AVC (ammonium acrylodimethyltaurate/VP copolymer) in de-ionized water;
Sample 3: an aqueous composition containing 4 wt % of acrylates copolymer in de-ionized water;
Sample 4: an aqueous composition containing 0.5 wt % of cellulose gum in de-ionized water;
Sample 5: an aqueous composition containing 4 wt % of Syntran PC5100 NP (water/ammonium acrylate copolymer/1,3-butanediol/C₁₁-C₁₅ pareth-7/sodium laureth-12sulfate/phenoxyethanol) in de-ionized water;
Sample 6: an aqueous composition containing 2.4 wt % of KGel (hydrolyzed keratin/water/phenoxyethanol) in de-ionized water;
Sample Blend: an aqueous composition containing a blend of all the ingredients of Samples 1-6 (i.e., PVP, Aristoflex AVC, acrylates copolymer, cellulose gum, Syntran PC5100 NP, and KGel) at the above-specified amounts in balancing de-ionized water;
Sample 7: a skin-tightening product commercially available under the registered trademark “LIFTFUSION” from Fusion Beauty Inc. at Ottawa, Canada;
Sample 8: another skin-tightening product commercially available under the trademark “NO-LINES” from Boyd's of Madison Avenue, Inc. at New York, N.Y.; and
Sample Taut: a topical composition containing ingredients as specified hereinabove in Formula VI.

Each of the above-listed sample compositions was applied onto the forearm of a subject in approximately the same amount, and measurements were conducted about ten (10) minutes after the application. Measuring mode 1 of the Cutometer® MPA 580 was used, in which the skin coated with each sample composition was drawn into an aperture of the probe with a constant negative pressure of about 450 millibars (approximately 6.53 pounds/square inch). After about 2 seconds, the negative pressure was switched off, and the skin was allowed to return to its original shape for about 2 seconds. The entire test was conducted at a constant temperature of about 20° C.

The Cutometer® MPA 580 generated a curve for each sample composition, indicative of the viscoelastic qualities of the skin. Specifically, the Cutometer® MPA 580 provides readings of: (a) the degree of skin distension (Uf) induced by application of the negative pressure, and (b) the degree of skin deformation (Ua) remained after the negative pressure is removed. The skin firmness, which is indicative of the resistance exhibited by the skin against the suction power generated by the negative pressure, is therefore calculated as 1/Uf, i.e., inverse to the degree of skin distension (Uf) as measured by the Cutometer® MPA 580 during the application of the negative pressure. The skin tonicity, which is indicative of the ability of the skin to return to its original state after the negative pressure is removed, is therefore calculated as 1/Ua, i.e., inverse the degree of skin deformation (Ua) as measured by the Cutometer® MPA 580 after the negative pressure is removed.

Additional calculation was carried out in order to provide comparative data showing the relative skin-firming and skin-toning effects of the various sample compositions. Specifically, the skin firmness before application of each sample composition was recorded as “Fb,” and the skin firmness after application of each sample composition was designated as “Fa.” The skin-firming effect (F) of each sample composition was then calculated as (Fa−Fb)/Fb. Similarly, the skin tonicity before application of each sample composition was designated as “Tb,” and the skin tonicity after application of the sample composition was designated as “Ta.” The skin-toning effect (T) of each sample composition was then calculated as (Ta−Tb)/Tb.

Further, averages of various parameters as described hereinabove were calculated for Samples 1-6 and then compared with the parameters of Sample Blend, which contained a blend of various ingredients that were individually provided in Samples 1-6.

The following table contains the experimental data obtained from the above-described skin firmness and skin tonicity tests:

TABLE I

| Sample No. | Uf | 1/Uf | F | Ua | 1/Ua | T |
|---|---|---|---|---|---|---|
| Control (clean skin) | 0.31 | 3.2258 | — | 0.24 | 4.1667 | — |
| 1 | 0.46 | 2.1739 | −33% | 0.28 | 3.5714 | −14% |
| 2 | 0.37 | 2.7027 | −16% | 0.29 | 3.4483 | −17% |
| 3 | 0.38 | 2.6316 | −18% | 0.29 | 3.4483 | −17% |
| 4 | 0.43 | 2.3256 | −28% | 0.33 | 3.0303 | −27% |
| 5 | 0.34 | 2.9412 | −8.8% | 0.28 | 3.5714 | −14% |
| 6 | 0.26 | 3.8462 | 19% | 0.20 | 5.0000 | 20% |
| Average 1–6 | 0.37 | 2.7027 | −16% | 0.27 | 3.7037 | −11% |
| Blend | 0.19 | 5.2632 | 63% | 0.11 | 9.0909 | 118% |
| 7 | 0.31 | 3.2258 | 0% | 0.25 | 4.0000 | −4% |
| 8 | 0.30 | 3.3333 | 3.3% | 0.18 | 5.5556 | 33% |
| Taut | 0.19 | 5.2632 | 63% | 0.08 | 12.5000 | 200% |

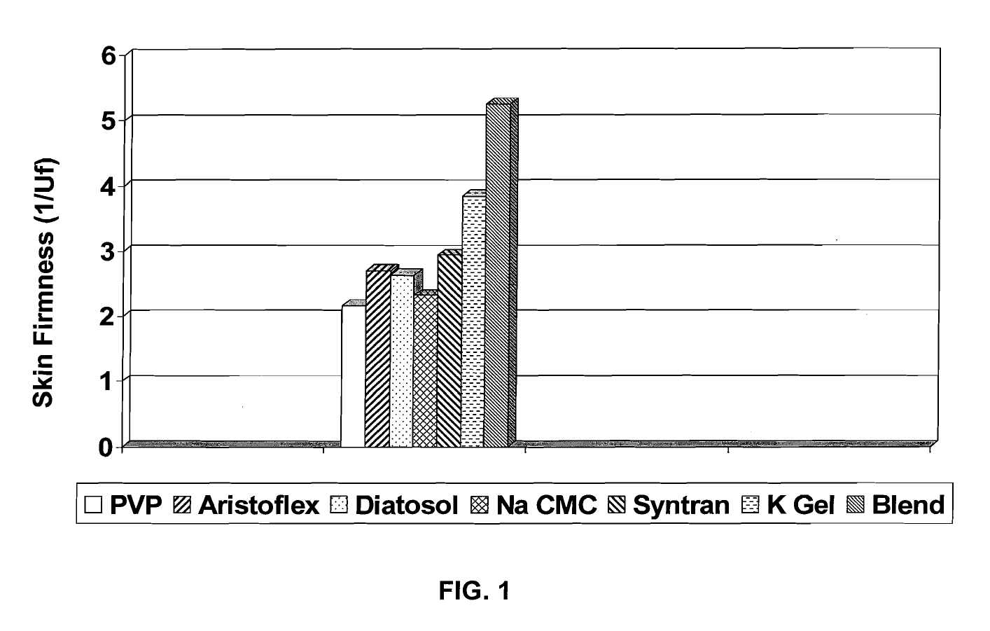

FIGS. 1-4 provide bar charts of the skin firmness (1/Uf) and skin tonicity (1/Ua) data, so as to better illustrate the improvements exhibited by Sample Blend and Sample Taut over Samples 1-6 and 7-8.

Specifically, FIG. 1 shows the skin firmness (1/Uf) observed after application of Sample Blend (which was formulated according to one embodiment of the present invention), in comparison with the skin firmness observed after application of Samples 1-6 (which contained various individual components of Sample Blend). It is clear from FIG. 1 that the skin firmness exhibited by Sample Blend is significantly higher than that exhibited by each of Samples 1-6. Further, the skin firmness exhibited by Sample Blend is significantly higher than the average skin firmness of Samples 1-6, as shown in Table 1. Therefore, Sample Blend has demonstrated a synergistic effect on the skin firmness, which was surprising and unexpected from the skin firmness data of the individual components as contained by Sample Blend.

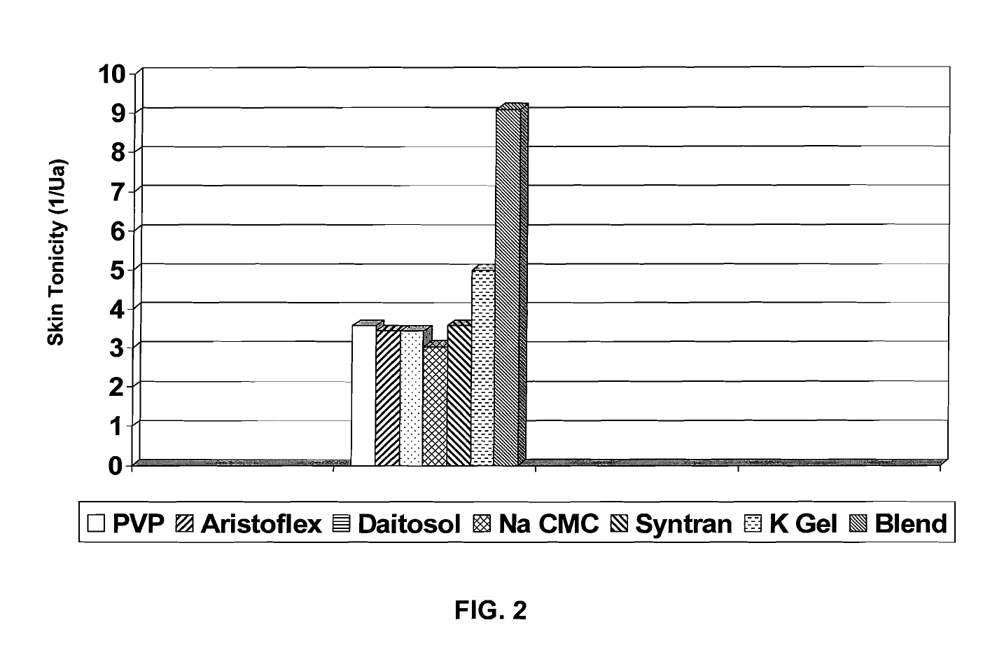

FIG. 2 shows the skin tonicity (1/Ua) observed after application of Sample Blend, in comparison with those observed after application of Samples 1-6. It is clear from FIG. 2 that the skin tonicity exhibited by Sample Blend is significantly higher than that exhibited by each of Samples 1-6. Further, the skin tonicity exhibited by Sample Blend is significantly higher than the average skin tonicity of Samples 1-6. Therefore, Sample Blend has also demonstrated a synergistic effect on the skin tonicity, which was surprising and unexpected from the skin firmness data of the individual components as contained by Sample Blend.

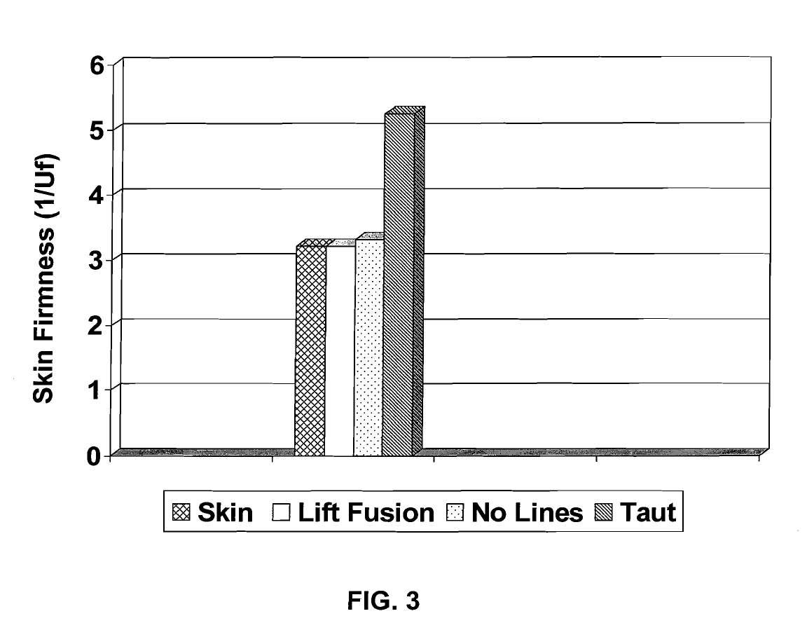

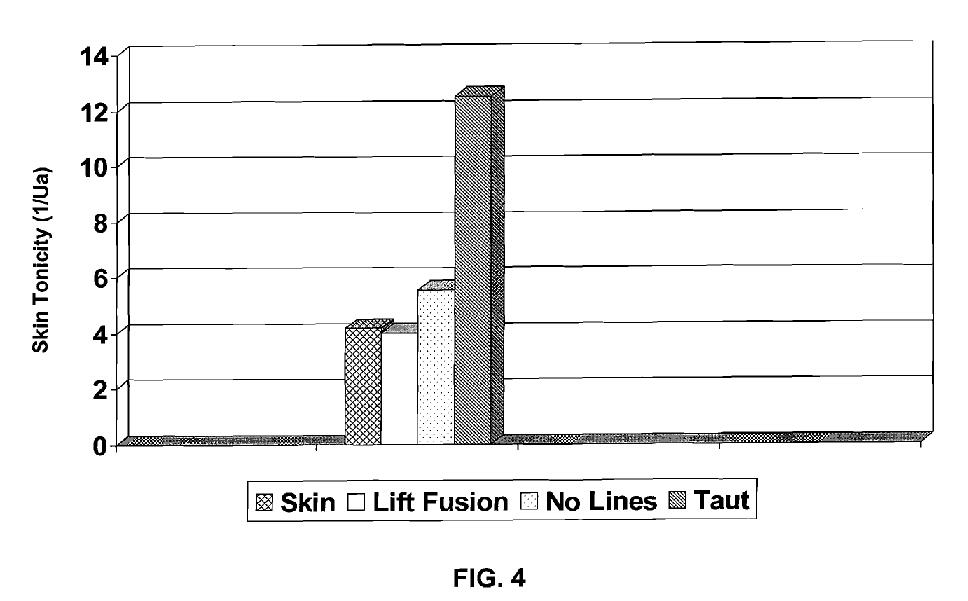

FIGS. 3-4 respectively illustrate the skin firmness (1/Uf) and the skin tonicity (1/Ua) observed after application of Sample Taut (which was formulated according to Formula VI of the present invention), in comparison with those observed for clean skin (Control) and those observed after application of the two skin-tightening products that are currently available on the market (i.e., LIFTFUSION® in Sample 7 and NO-LINES™ in Sample 8). It is clear from FIGS. 3-4 that the skin firmness and skin tonicity exhibited by Sample Taut are significantly higher than that exhibited by the LIFTFUSION® and NO-LINES™ products.

### EXAMPLE 3

### Adhesion Test

Adhesion is the molecular attraction exerted between two bodies in contact. The adhesion of films formed by Samples 1-6 and Sample Blend was measured by a Texture Analyzer (model # TA XT PLUS) manufactured by Texture Technology Corporation at Scardale, N.Y. Specifically, a glass plate having a width of about 50 mm and a length of about 100 mm was used as a substrate. Each sample composition as described hereinabove (i.e., Samples 1-6 and Sample Blend) was cast down on the substrate to form a film of about 2 mils in thickness using a Bird type film applicator. In order to reduce the solvent content in certain films, the films were placed in an oven to effectuate better evaporation of the solvents. The glass plate with the film cast on one side was then placed onto a holder bracket of the texture analyzer along the vertical position. Next, a razor blade that was attached to a bracket at 60° angle, which was in turn attached to the shoulder of the texture analyzer, was placed onto the glass plate, and the texture analyzer was subsequently turned on. The razor blade scraped the film from the glass plate, and the amount of force used for peeling the film off the glass plate was then measured in grams as an indication of the adhesion of the film to the glass plate.

The following table provides the experimental results from the above-described adhesion tests:

TABLE 2

| Sample No. | Adhesion (g) |
|---|---|
| 1 | 1700 |
| 2 | 800 |
| 3 | 900 |
| 4 | 900 |
| 5 | 2500 |
| 6 | 1800 |
| Average 1–6 | 1430 |
| Blend | 1750 |

It is clear from TABLE 2 that the adhesion exhibited by Sample Blend is higher than the average adhesion of Samples 1-6 and is comparable with that exhibited by the PVP film. Therefore, Sample Blend demonstrated significantly enhanced skin-firming and skin-toning effects, but without compromising its adhesion to the skin. Correspondingly, Sample Blend can be used to form a flexibility film with good skin-adhesion for firming and toning the skin.

Although the invention has been variously disclosed herein with reference to illustrative embodiments and features, it will be appreciated that the embodiments and features described hereinabove are not intended to limit the scope of the invention, and that other variations, modifications and other embodiments will suggest themselves to those of ordinary skill in the art. The invention therefore is to be broadly construed, consistent with the claims hereafter set forth.

What is claimed is:

1. A topical composition for improving the appearance of the skin, comprising:
a water-soluble film-forming polymer;
a bimodal copolymer comprising a first polymeric component with anionic functional groups and a second polymeric component with cationic functional groups; and
one or more biological polymers that are derived from a source selected from the group consisting of animals, plants, algae, fungi, and bacteria or are biotechnologically synthesized.

2. The topical composition of claim 1, which forms a polymeric film on the skin after application thereto, wherein the polymeric film is characterized by a skin-firming effect of no less than about 30% and/or a skin-toning effect of no less than about 50%.

3. The topical composition of claim 2, wherein the polymeric film is characterized by a skin-firming effect of no less than about 50% and/or a skin-toning effect of no less than about 100%.

4. The topical composition of claim 2, wherein the polymeric film is characterized by a skin-firming effect of no less than about 60% and/or a skin-toning effect of no less than about 180%.

5. The topical composition of claim 2, wherein the polymeric film is further characterized by an adhesion of at least 1500 grams.

6. The topical composition of claim 1, wherein the water-soluble film-forming polymer is selected from the group consisting of polyvinylpyrrolidone (PVP), polyvinylacetate (PVA), PVP/PVA copolymers, carboxymethylcellulose (CMC), cellulosic ethers, polyamides, acrylates polymers and copolymers, PVP/dimethylaminopropylamine (DMAPA) acrylates copolymers, vinylpyrrolidone (VP)/lauryl methacrylate (LMA) copolymers, ammonium acrylodimethyltaurateNVP copolymers, isobutylene (IB)/maleic anhydride (MA) copolymers, imidized IB/MA copolymers, acrylates/acrylonitrogen copolymers, polysiloxanes, silicon-containing polymers, and combinations thereof.

7. The topical composition of claim 6, comprising from about 1% to about 10% of the water-soluble film-forming polymer by total weight of said composition.

8. The topical composition of claim 1, wherein the first polymeric component of the bimodal copolymer comprises one or more monomeric units having the formula of: 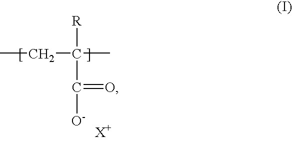
wherein R is hydrogen or an alkyl group and X⁺ is a salt-forming cation.

9. The topical composition of claim 8, wherein the second polymeric component of the bimodal copolymer comprises one or more monomeric units having the formula of: 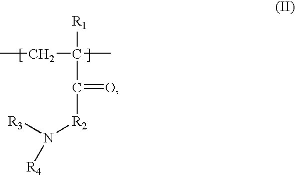
or a quatemized adduct thereof, wherein R₁, R₃ and R₄ are, independently, hydrogen or an alkyl group and R₂ is an alkyl group.

10. The topical composition of claim 9, wherein the first polymeric component comprises one or more monomeric acrylate units, and wherein the second polymeric component comprises one or more monomeric dialkylaminoalkylmethacrylate units or quatemized adducts thereof.

11. The topical composition of claim 10, comprising from about 1% to about 10% of the bimodal copolymer by total weight of said composition.

12. The topical composition of claim 1, wherein said one or more biological polymers are selected from the group consisting of keratin, cellulose gum, alginates, carrageenans, sweet almond proteins, argan proteins, corn proteins, wheat proteins, silk proteins, milk proteins, soy proteins, tobacco leaf proteins, bacterial proteins, fibrous proteins derived from beans, albumins, and bacterial polysaccharides.

13. The topical composition of claim 12, comprising two or more biological polymers selected from the group consisting of keratin, cellulose gum, alginates, and carrageenans.

14. The topical composition of claim 12, comprising from about 0.1% to about 30% of said biological polymers by total weight of said composition.

15. The topical composition of claim 1, further comprising one or more skin care additives.

16. The topical composition of claim 15, wherein said one or more skin care additives are selected from the group consisting of anti-aging agents, anti-acne agents, enzyme-inhibiting agents, collagen-stimulating agents, sunscreen agents, antioxidants, and exfoliants.

17. A method of enhancing the appearance of skin, comprising applying to the skin a topical composition comprising: (1) a water-soluble film-forming polymer, (2) a bimodal copolymer comprising a first polymeric component with anionic functional groups and a second polymeric component with cationic functional groups, and (3) one or more biological polymers that are derived from a source selected from the group consisting of animals, plants, algae, fungi, and bacteria or are biotechnologically synthesized.

18. The method of claim 17, wherein the topical composition is applied to saggy or wrinkled skin.

19. The method of claim 18, wherein the topical composition is applied as needed to the saggy or wrinkled skin to achieve immediate wrinkle reduction.

20. The method of claim 17, wherein the topical composition forms a polymeric film on the skin after application thereto, wherein the polymeric film is characterized by a skin-firming effect of no less than about 30% and a skin-toning effect of no less than about 50%.

21. The method of claim 20, wherein the polymeric film is characterized by a skin-firming effect of no less than about 50% and/or a skin-toning effect of no less than about 100%.

22. The method of claim 20, wherein the polymeric film is characterized by a skin-firming effect of no less than about 60% and/or a skin-toning effect of no less than about 180%.

23. The method of claim 20, wherein the polymeric film is further characterized by an adhesion of no less than about 1500 grams.

24. The method of claim 18, wherein the water-soluble film-forming polymer is selected from the group consisting of polyvinylpyrrolidone (PVP), polyvinylacetate (PVA), PVP/PVA copolymers, carboxymethylcellulose (CMC), cellulosic ethers, polyamides, acrylates polymers and copolymers, PVP/dimethylaminopropylamine (DMAPA) acrylates copolymers, vinylpyrrolidone (VP)/lauryl methacrylate (LMA) copolymers, ammonium acrylodimethyltaurate/VP copolymers, isobutylene (IB)/maleic anhydride (MA) copolymers, imidized IB/MA copolymers, acrylates/acrylonitrogen copolymers, polysiloxanes, silicon-containing polymers, and combinations thereof.

25. The method of claim 24, wherein the topical composition comprises from about 1% to about 10% of the water-soluble film-forming polymer by total weight of said composition.

26. The method of claim 18, wherein the first polymeric component of the bimodal copolymer comprises one or more monomeric units having the formula of: 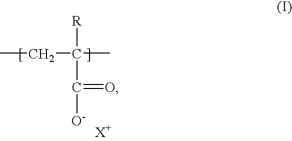
wherein R is hydrogen or an alkyl group and X⁺ is a salt-forming cation.

27. The method of claim 26, wherein the second polymeric component of the bimodal copolymer comprises one or more monomeric units having the formula of: 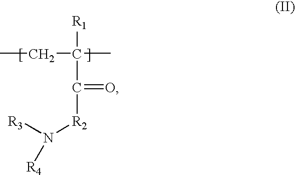
or a quatemized adduct thereof, wherein R₁, R₃ and R₄ are, independently, hydrogen or an alkyl group and R₂ is an alkyl group.

28. The method of claim 26, wherein the first polymeric component comprises one or more monomeric acrylate units, and wherein the second polymeric component comprises one or more monomeric dialkylaminoalkylmethacrylate units or quatemized adducts thereof.

29. The method of claim 27, wherein the topical composition comprises from about 1% to about 10% of the bimodal copolymer by total weight of said composition.

30. The method of claim 18, wherein said one or more biological polymers are selected from the group consisting of keratin, cellulose gum, alginates, carrageenans, sweet almond proteins, argan proteins, corn proteins, wheat proteins, silk proteins, milk proteins, soy proteins, tobacco leaf proteins, bacterial proteins, fibrous proteins derived from beans, albumins, and bacterial polysaccharides.

31. The method of claim 30, wherein the topical composition comprises two or more biological polymers selected from the group consisting of keratin, cellulose gum, alginates, and carrageenans.

32. The method of claim 30, wherein the topical composition comprises comprising from about 0.1% to about 30% of said biological polymers by total weight of said composition.

33. The method of claim 18, wherein the topical composition further comprising one or more skin care additives.

34. The method of claim 33, wherein the skin care additives are selected from the group consisting of anti-aging agents, anti-acne agents, enzyme-inhibiting agents, collagen-stimulating agents, sunscreen agents, antioxidants, and exfoliants.
